(19) United States
(12) Patent Application Publication
(10) Pub. No.: US 2026/0199335 A1
(43) Pub. Date: Jul. 16, 2026

(54) METHODS FOR TREATING PATIENTS WITH HEMATOLOGIC MALIGNANCIES

(71) Applicant: Aptose Biosciences Inc., Mississauga (CA)

(72) Inventors: Rafael BEJAR, La Jolla, CA (US); William G. RICE, Del Mar, CA (US); Young Su NOH, Hwaseong-si (KR); Jiyeon YOON, Hwaseong-si (KR); Eunhye BAEK, Hwaseong-si (KR); Sooa JUNG, Hwaseong-si (KR)

(21) Appl. No.: 19/137,415

(22) PCT Filed: Dec. 8, 2023
(86) PCT No.: PCT/US2023/083227

§ 371 Date: Jun. 10, 2025

Related U.S. Application Data
(60) Provisional application No. 63/431,693, filed on Dec. 10, 2022.

(51) Int. Cl. A61K 31/506; A61K 9/00; A61K 31/44; A61K 31/4709; A61K 31/497; A61K 31/5377; A61K 31/553; A61K 31/635; A61K 31/706; A61P 35/02

(52) U.S. Cl. CPC A61K 31/506; A61K 9/0053; A61K 31/44; A61K 31/4709; A61K 31/497; A61K 31/5377; A61K 31/553; A61K 31/635; A61K 31/706; A61P 35/02

(57) ABSTRACT

The present disclosure relates to compounds of Formula 1 and pharmaceutical compositions thereof for the treatment of cancer in a subject having a mutant form of NPM1, DNMT3A, RAS, or a combination thereof, or for specific treatments of subjects with relapsed or treatment-refractory (R/R) acute myeloid leukemia (AML).

### CROSS-REFERENCE TO RELATED APPLICATIONS

This application claims priority to U.S. Provisional Application No. 63/431,693, filed Dec. 10, 2022, the disclosure of which is herein incorporated by reference in its entirety for all purposes.

### BACKGROUND OF THE INVENTION

Acute myeloid leukemia (AML) is an aggressive hematologic disorder in which the hematopoietic stem cells lose their ability to differentiate normally and continue to proliferate. It carries a poor prognosis and has garnered few treatments advanced in the last few decades. Multiple abnormalities including FMS-like tyrosine kinase-3 (FLT3), and nucleophosmin (NPM1), rat sarcoma (RAS) have been observed in patients with AML. For example, the RAS oncogenes, NRAS and KRAS are frequently mutated in AML, occurring in 11% and 5% of patients, whereas FLT3 gene mutations are found in approximately 30% of adult AML. Activating mutations in FLT3, such as Internal Tandem Duplication (ITD) in the proximal domain, account for about 25-30% of newly diagnosed AML cases and are associated with poor prognosis. (British Journal of Hematology, 2003, 122, 523-538). It is known that FLT3 mutations occur in about ⅓ of patients with acute myeloid leukemia (AML). In addition, although there are several clinically available FLT3 inhibitors, drug-resistant leukocyte cells were observed in AML patients treated with these FLT3 inhibitors, indicating drug resistance (Cancer Science 2020 Volume 111:312-322). Additionally, conventional Acute Myeloid Leukemia (AML) standard chemotherapy cannot target AML stem/progenitor cells, which frequently causes disease recurrence in patients, thereby limiting long-term efficacy (Oncogene 2010 Volume 29: 5120-5134). Therefore, there is a need for compounds, compositions, and methods that can effectively treat patients with hematological malignancies such as mutated AML harboring mutations such as NPM1, DNMT3A, RAS, or a combination thereof.

### SUMMARY OF THE INVENTION

In embodiments, the present disclosure provides compounds, pharmaceutical compositions, and methods for the treatment of cancer e.g., acute myeloid leukemia in a subject with one or more mutations (e.g., as defined herein) by the use of a compound of Formula (1), or a pharmaceutically acceptable salt thereof, solvate thereof, stereoisomer thereof, tautomer thereof, or combination thereof.

In embodiments, the present disclosure provides methods of treating cancer in a subject in need thereof, comprising administering to the subject a therapeutically effective amount of a compound of Formula (1), or a pharmaceutically acceptable salt thereof, solvate thereof, stereoisomer thereof, tautomer thereof, or combination thereof, wherein the subject has a mutant form of NPM1, DNMT3A, RAS, or a combination thereof:

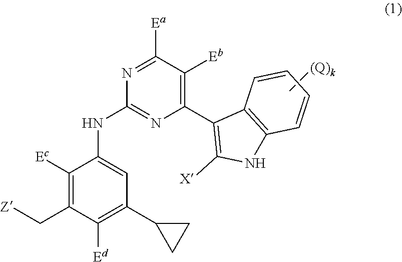

wherein in Formula (1):
Ea is hydrogen, hydroxy or C1-4 alkoxy;
Eb is hydrogen, halogen, C1-4 alkyl or C1-4 fluoroalkyl;
Ec and Ed are each independently hydrogen or hydroxy;
X′ is hydrogen or hydroxy;
k is an integer from 1 to 2;
each Q is independently hydroxy, halogen, C1-4 alkyl, hydroxy C1-4 alkyl or C1-4 alkoxy;
Z′ is a monovalent functional group represented by Formula (2);

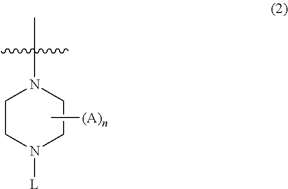

wherein:
each A is independently selected from the group consisting of hydroxy, C1-4 alkyl and hydroxy C1-4 alkyl, wherein at least one A is C1-4 alkyl;
n is an integer from 1 to 2; and
L is hydrogen, C1-4 alkyl, hydroxy or hydroxy C1-4 alkyl.

In embodiments, the compound of Formula (1) is a compound of Formula (3), or a pharmaceutically acceptable salt thereof, solvate thereof, stereoisomer thereof, tautomer thereof, or combination thereof;

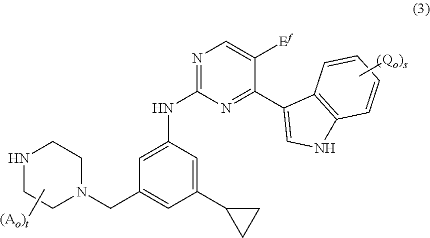

wherein in Formula 3:
Ef is fluorine, chlorine, bromine or iodine;
Qo is hydroxy, halogen, C1-4 alkyl, hydroxy C1-4 alkyl or C1-4 alkoxy;
s is an integer from 1 to 2;
Ao is selected from the group consisting of hydroxy, C1-4 alkyl and hydroxy C1-4 alkyl; and
t is an integer from 1 to 2.

In embodiments, the compound of Formula (1) is Compound I

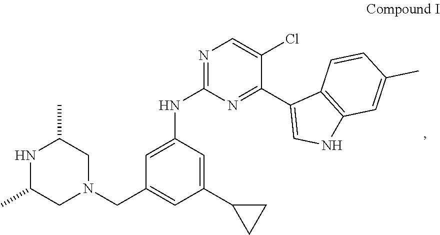

or a pharmaceutically acceptable salt thereof, solvate thereof, stereoisomer thereof, tautomer thereof, or combination thereof.

In embodiments, the subject has a mutant form of NPM1, DNMT3A, RAS, or a combination thereof and additionally has a mutant form of FLT3.

In embodiments, the cancer is a hematological cancer such as leukemia.

In embodiments, the leukemia is acute myeloid leukemia (AML).

### BRIEF DESCRIPTION OF THE DRAWINGS

FIG. 1 shows clinical responses of various patients treated with Compound I.

FIG. 2 shows a treatment protocol for the combination of Compound I and Venetoclax.

FIG. 3 shows clinical responses of various patients treated with the combination of Compound I and Venetoclax.

### DETAMLED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

While the following terms are believed to be well understood by one of ordinary skill in the art, the following definitions are set forth to facilitate explanation of the presently disclosed subject matter.

Throughout the present specification, the terms “about” and/or “approximately” can be used in conjunction with numerical values and/or ranges. The term “about” is understood to mean those values near to a recited value. For example, “about 40 [units]” can mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or therebelow. Furthermore, the phrases “less than about [a value]” or “greater than about [a value]” should be understood in view of the definition of the term “about” provided herein. The terms “about” and “approximately” can be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range can be an endpoint for the range encompassed thereby (e.g., the range 50-80 includes the ranges with endpoints such as 55-80, 50-70, etc.).

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

In the following description, certain specific details are set forth in order to provide a thorough understanding of various embodiments. However, one skilled in the art will understand that the disclosure can be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Administration in “conjunction with” another therapeutically active agent includes administration in the same or different composition(s) and/or combinations, either sequentially, simultaneously, or continuously, through the same or different routes. In some embodiments, the combination therapy optionally includes one or more pharmaceutically acceptable carriers or excipients, non-pharmaceutically active compounds, and/or inert substances.

The term “combination therapy” refers to a first therapy that includes an active agent, for example, Compound I, in conjunction with one or more therapeutically active agents useful for treating, stabilizing, preventing, and/or delaying the disease or condition. The terms “pharmaceutical combination,” “therapeutic combination” or “combination” as used herein, refers to a single dosage form comprising at least two therapeutically active agents, or separate dosage forms comprising at least two therapeutically active agents together or separately for use in a combination therapy. For example, one therapeutically active agent may be formulated into one dosage form or composition and the other therapeutically active agent may be formulated into a single or different dosage form or composition. In a specific example, one therapeutically active agent may be formulated into a solid oral dosage form or composition whereas the second therapeutically active agent may be formulated into another oral dosage form or composition, including as a kit, or from two kits.

The term “halogen” includes fluorine, chlorine, bromine or iodine, unless otherwise indicated, and may be, for example, fluorine or chlorine, but is not limited thereto.

The term “alkyl” refers to a saturated monovalent hydrocarbon radical. The term “alkenyl” used herein refers to a monovalent hydrocarbon radical containing at least one carbon-carbon double bond, wherein each double bond may have an E-or Z-steric configuration. The term “alkynyl” used herein refers to a monovalent hydrocarbon radical containing at least one carbon-carbon triple bond. Such an alkyl group, an alkenyl group, and an alkynyl group may be linear, i.e., straight-or side-chained. As defined above, the number of carbon atoms in an alkyl group may be 1, 2, 3, 4, 5, or 6; or 1, 2, 3, or 4. Examples of alkyl include methyl, ethyl, propyl including n-propyl and iso-propyl, n-butyl, sec-butyl, butyl including iso-butyl and a tert-butyl, pentyl including n-pentyl, 1-methylbutyl, iso-pentyl, neo-pentyl, and tert-pentyl, hexyl including n-hexyl, 3,3-dimethylbutyl, and iso-hexyl. A double bond of an alkenyl group and a triple bond of an alkynyl group may each be in any position. Examples of alkenyl and alkynyl are ethenyl, prop-1-enyl, prop-2-enyl (=allyl), but-2-enyl, 2-methylprop-2-enyl, 3-methylbut-2-enyl, hex-3-enyl, hex-4-enyl, prop-2-enyl(=propagyl), but-2-enyl, but-3-enyl, hex-4-enyl, and hex-5-enyl. In a case where each of the compounds is sufficiently stable and suitable for a desirable use as, for example, a pharmaceutical substance, a substituted alkyl group, a substituted alkenyl group, and a substituted alkynyl group may be substituted at any position.

The term “cycloalkyl”, unless otherwise stated, refers to a substituted or unsubstituted cyclic alkyl group, and an example of a single or multi-cyclic group is a mono-or bicycloaliphatic group. Examples of a cycloalkyl group include cyclopropyl, cyclobutyl, cyclopentyl, cyclohexyl, cyclohexenyl, cycloheptyl, cycloheptenyl, cyclooctyl, cyclooctenyl, 2,5-cyclohexadienyl, bicyclo[2.2.2]octyl, adamant-1-yl, decahydronaphthyl group, oxo cyclohexyl, dioxo cyclohexyl, thio cyclohexyl, 2-oxo bicyclo[2.2.1]hept-1-yl, or any suitable isomer thereof without limitation.

The term “heterocycloalkyl” used herein, unless otherwise stated, refers to a substituted or unsubstituted mono cyclic or multicyclic alkyl containing at least one selected from O, N, and S, for example, 1 to 4 heteroatoms Examples of the mono heterocyclo alkyl group are piperazinyl, piperidinyl, piperazinyl-1-oxide, morpholinyl, thiamorpholinyl, pyrrolidinyl, imidazolidinyl, tetrahydrofuranyl, diazabicycloheptanyl, diazabicyclooctane, and diazaspirooctane, and groups similar thereto, but are not limited thereto.

The term “heterobicycloalkyl” used herein, unless otherwise stated, refers to a bicycloalkyl containing one or more hetero atom selected from O, N, and S, and includes a fused hetero bicycloalkyl group and a bridged hetero bicycloalkyl group. The term “bridged” used herein refers to a valence bond, single atom, or non-branched chain of atoms that connects two different parts inside a molecule. Also, a pair of tertiary or more carbon atoms linked through bridges is called “bridge heads.” In other words, the carbon atoms that are simultaneously participating as a part of two or more rings are called bridge heads, and bonds connected to these bridge heads are called bridges. The term “bridged compound” used herein refers to a compound in which two or more rings share one or more pairs of carbon atoms.

Examples of a fused heterobicycloalkyl group include indole, quinoline, thiazolo[4,5-b]-pyridine, quinoline, and the like, but are not limited thereto. Examples of bridged heterobicycloalkyl include 7 to 12 membered heterobicycloalkyls such as a diazabicyclo [2.2.1]heptane or a diazabicyclo [3.2.1]octane, but are not limited thereto.

The term “spiro” used herein refers to, unless defined otherwise, two rings which share one atom, wherein the two rings are not connected to each other by bridges. The term “spirocycloalkyl” used herein refers to, unless defined otherwise, a saturated carbocycle consisting of two rings, which share only one carbon atom as part of the rings. Examples of spirocycloalkyl include a 7 to 12 membered spirocycloalkyl group such as diazaspiro[2.5]octane, but are not limited thereto. The term “heterospirocycloalkyl” refers to, unless defined otherwise, a spirocycloalkyl containing at least one heteroatom selected from O, N, and S. The expression“spiro connection” used herein refers to a linker sharing one atom, unless defined otherwise.

The term “aryl” used herein, unless otherwise stated, refers to an aromatic group which may be substituted or unsubstituted, such as phenyl, biphenyl, naphthyl, toluyl, naphthalenyl, anthracenyl, or any suitable isomer thereof without limitation.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

As used herein, the term “solvate” is used to describe a molecular complex, which may exist as a compound according to the present invention and one or more pharmaceutically acceptable solvent molecules. It refers to a molecular complex of a compound of the present invention (or a pharmaceutically acceptable salt thereof) with one or more solvent molecules. Such solvent molecules may be those known or commonly used in the pharmaceutical art, for example, water, ethanol, and the like. The term “hydrate” refers to a complex in which the solvent molecule is water.

In embodiments, the therapeutic drug in the pharmaceutical composition may be provided in the form of a “solvate”, wherein the solvate includes a hydrate.

The term “stereoisomer” used herein refers to a compound that has the same molecular formula and the same connection order of atoms and is different in stereoscopic or optical aspects. That is, a stereoisomer refers to a compound having the same chemical composition but being different in the three-dimensional arrangement aspect, that is, being different in the arrangement of atoms or groups. The stereoisomer includes geometric isomers, enantiomeric isomers, and partial stereoisomers.

The term “geometrical isomer” used herein refers to the type of a stereoisomer depending on the direction of a functional group in a molecule, and may be called a cis-trans isomer. Generally, these isomers contain non-rotatable double bonds and the substituents of compounds containing double bonds may be in an E form or a Z form. For example, when the compound contains a 2-substituted cycloalkyl group, the compound may have the cis-trans form. When the compound of Formula 1 contains a bridged ring, the compound may exist as an exo or endo isomer.

The term “chiral” used herein refers to a molecule having a non-overlapping enantiomeric partner, and the term “achiral” used herein refers to a molecule having an overlapping enantiomeric partner. The term “enantiomer” used herein refers to the case where two molecules having optical activity are mirror-symmetrical. That is, the enantiomer indicates an isomer that does not overlap an original molecule, and does not have any of the symmetry elements including the symmetry plane and the symmetry center, and has the stereoscopic center (chiral center). The term “diastereomer” used herein refers to the case in which molecules having two or more chiral centers are not enantiomers but stereoisomers. Since the compounds of Formula 1 according to an aspect of the present disclosure may have chiral centers or asymmetric carbon centers (absent carbons), the compounds may exist as the enantiomer(R or S isomer), racemates, diastereoisomers, or a mixture thereof, and all of these isomers and mixtures are included within the scope of the present disclosure. The optically active (R)-and (S)-isomers may be decomposed by using techniques of the related art or chiral synthon or chiral reagents.

The term “constitutional isomers” used herein refers to isomers having the same molecular formula but different connection orders of atoms, and may include tautomer. The term “tautomers” used herein refers to constitutional isomers having different energy structures that are interchanged through low energy barriers. For example, a photon tautomer (also a proton tautomer) includes interconversion through the transfer of photons, such as keto-enol and imine-enamine isomerization. Valence tautomers include interconversions by the rearrangement of some electrons in the bound electrons. The compound of Formula 1 according to an aspect, and the stereoisomer or tautomer thereof may exist in the form of solvate.

A “pharmaceutical composition” refers to a formulation of a compound of the disclosure and a medium generally accepted in the art for the delivery of the biologically active compound to mammals, e.g., humans. Such a medium includes all pharmaceutically acceptable carriers, diluents or excipients therefor. The pharmaceutical composition of the present invention can be formulated according to a conventional method, and various oral dosage forms such as tablets, pills, powders, capsules, syrups, emulsions, microemulsions, or parenteral administration such as intramuscular, intravenous or subcutaneous administration can be prepared in dosage form.

When the pharmaceutical composition is prepared in the form of an oral dosage form, examples of carriers, additives, and excipients used include diluents, disintegrants, binders, lubricants, surfactants, suspending agents, or emulsifiers. When the pharmaceutical composition of the present invention is prepared in the form of an injection, the carrier or additive or excipient includes water, saline, aqueous glucose solution, similar sugar solution, alcohol, glycol, ether (eg, polyethylene glycol 400), oil, fatty acid, and fatty acid esters, glycerides, surfactants, suspending agents, or emulsifiers. Such formulation methods are well known to those skilled in the art of pharmaceuticals.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

An “effective amount” means the amount of a formulation according to the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated

The term “therapeutically effective” applied to dose or amount refers to that quantity of a compound or pharmaceutical formulation that is sufficient to result in a desired clinical benefit after administration to a patient in need thereof.

As used herein, a “subject” can be a human, non-human primate, mammal, rat, mouse, cow, horse, pig, sheep, goat, dog, cat and the like.

“Mammal” includes humans and both domestic animals such as laboratory animals (e.g., mice, rats, monkeys, dogs, etc.) and household pets (e.g., cats, dogs, swine, cattle, sheep, goats, horses, rabbits), and non-domestic animals such as wildlife and the like.

### Therapeutic Use

In embodiments, the present disclosure provides compounds, pharmaceutical compositions, and methods for the treatment of cancer e.g., acute myeloid leukemia in a subject with one or more mutations (e.g., as defined herein) by the use of a compound of the present disclosure (e.g., a compound of Formula (1), Formula (3), Compound A, or Compound I) or a pharmaceutically acceptable salt thereof, solvate thereof, stereoisomer thereof, tautomer thereof, or combination thereof.

Compounds of the present disclosure, compositions and uses thereof are described, for example, in U.S. Pat. Nos. 10,870,639, 11,292,786, International Application No. PCT/KR2021/015794 (Published as WO2022/098083), and International Application No. PCT/KR2022/016095 the contents of each of which is hereby incorporated by reference their entireties for all purposes.

In embodiments, provided herein are methods of treating cancer in a subject in need thereof, comprising administering to the subject a therapeutically effective amount of a compound of Formula (1), or a pharmaceutically acceptable salt thereof, solvate thereof, stereoisomer thereof, tautomer thereof, or combination thereof, wherein the subject has a mutant form of NPM1, DNMT3A, RAS, or a combination thereof:

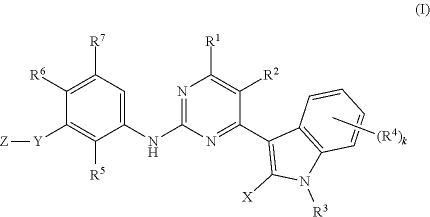

Wherein:
R¹ is hydrogen, a halogen, a hydroxy group, a C₁-C₄ alkoxy group, or —NRₐR_{b},
wherein Rₐ and R_{b} are each independently hydrogen or a C₁-C₄ alkyl group;
R² is hydrogen, a halogen, a cyano group, a nitro group, an amino group, a carboxamide group, a formyl group, a halo C₁-C₄ alkyl group, or a C₁-C₄ alkyl group;
R³ is hydrogen, a halogen, a hydroxy group, a halo C₁-C₄ alkyl group, a C₁-C₄ alkyl group, a C₂-C₄ alkenyl group, or a C₂-C₄ alkynyl group;
Each R⁴ is independently hydrogen, a halogen, a hydroxy group, a cyano group, a nitro group, an amino group, —S(═O)₁—R_{c}, a halo C₁-C₄ alkyl group, a C₁-C₄ alkoxy group, a hydroxy C₁-C₄ alkyl group, a C₁-C₄ alkyl group, a C2-C4 alkenyl group, a C2-C4 alkynyl group, —NR_{d}Rₑ, —CO₂Rₑ, or —CO—NR_{d}Rₑ,
wherein Rₑ is a C₁-C₄ alkyl group or —NR_{d}Rₑ,
R_{d} and Rₑ are each independently hydrogen or a C₁-C₄ alkyl group,
I is an integer from 0 to 2, and
k is an integer from 0 to 4;
R⁵ and R⁶ are each independently hydrogen, a halogen, a hydroxy group, a nitro group, an amino group, a C₁-C₄ alkoxy group, or a C₁-C₄ alkyl group;
R⁷ is a hydroxy C₁-C₄ alkyl group, a C₁-C₄ alkyl group, a C2-C4 alkenyl group, a C2-C4 alkynyl group, a C₃-C₇ cycloalkyl group, or a C3-C9 heterocycloalkyl group,
wherein the C₃-C₇ cycloalkyl group or the C₃-C₉ heterocycloalkyl is unsubstituted or substituted with a halogen, a C₁-C₄ alkyl group, or a halo C₁-C₄ alkyl group; and
X is H or OH;
wherein, when X is OH, the compound represented by Formula 1 includes a tautomeric structure represented by Formula 2,

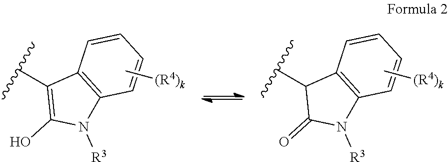

R³, R⁴, and k in Formula 2 are the same as described in connection with Formula 1; Y is —(CH₂)ₘ—, —(CH₂)ₘ—O—(CH₂)ₙ—, —(CH₂)ₘ—CO—(CH₂)ₙ—, —(CH₂)ₘ—NR—(CH₂)ₙ—, or —(CH₂)ₘ—SO₂—(CH₂)ₙ—,
wherein R^{g} is hydrogen or a C₁-C₄ alkyl group,
m and n are each independently an integer from 0 to 2; and
Z is represented by Formula 3;

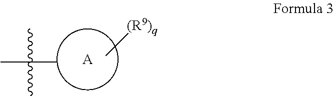

wherein, in Formula 3,
\textcircled{A} is a C₃-C₁₀ cycloalkyl group or a C₂-C₁₁ heterocycloalkyl group;
R⁹ is a halogen, a hydroxy group, a cyano group, a nitro group, an amino group, a thiol group, a formyl group, a halo C₁-C₄ alkyl group, a C₁-C₄ alkoxy group, a linear or branched hydroxy C₁-C₄ alkyl group, a linear or branched C₁-C₄ alkyl group, a C₂-C₄ alkenyl group, a C₂-C₄ alkynyl group, a C₃-C₁₀ cycloalkyl group, a C₂-C₉ heterocycloalkyl group, a hydroxy C₂-C₉ heterocycloalkyl group, a linear or branched hydroxy C₁-C₄ alkylcarbonyl group, —NR¹⁰R¹¹, —COR¹², —COOR¹², or —SO₂R¹³,
q is an integer from 0 to 5,
wherein, when \textcircled{A} is piperazine or piperidine, q is not 0, and
wherein two or more R⁹ are connected or fused with \textcircled{A} to form a 7- to 12-membered bicycloalkyl group, heterobicycloalkyl group, spirocycloalkyl group, or spiroheterocycloalkyl group;
R¹⁰ and R¹¹ are each independently hydrogen, a hydroxy C₁-C₄ alkyl group, a halo C₁-C₄ alkyl group, a C₁-C₄ alkyl group, a C₂-C₄ alkenyl group, or a C₂-C₄ alkynyl group;
R¹² is hydrogen, a hydroxy group, a hydroxy C₁-C₄ alkyl group, a halo C₁-C₄ alkyl group, a C₁-C₄ alkyl group, a C₂-C₄ alkenyl group, a C₂-C₄ alkynyl group, a C₃-C₁₀ cycloalkyl group, or a C₂-C₉ heterocycloalkyl group;
R¹³ is hydroxy, a halo C₁-C₄ alkyl group, a C₁-C₄ alkyl group, a C₂-C₄ alkenyl group, a C₂-C₄ alkynyl group, a C₃-C₁₀ cycloalkyl group, a C₂-C₉ heterocycloalkyl group, aryl group, or —NR_{f}R_{g}, and
R_{f} and R_{g} are each independently hydrogen or a C₁-C₄ alkyl group.

In embodiments, provided herein are methods of treating cancer in a subject in need thereof, comprising administering to the subject a therapeutically effective amount of a compound of Formula (14), or a pharmaceutically acceptable salt thereof, solvate thereof, stereoisomer thereof, tautomer thereof, or combination thereof, wherein the subject has a mutant form of NPM1, DNMT3A, RAS, or a combination thereof:

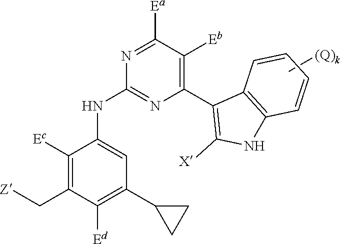

Wherein:
E^{a} is hydrogen, a hydroxy, or a C₁-C₄ alkoxy group;
E^{b} is hydrogen, a halogen, a C₁-C₄ alkyl group, or a C₁-C₄ fluoroalkyl group;
E^{c} and E^{d} are each independently hydrogen or a hydroxy group;
X′ is hydrogen or a hydroxy group;
k is an integer from 0 to 4;
each Q is independently hydroxy, a halogen, a C₁-C₄ alkyl group, a hydroxy C₁-C₄ alkyl group, or a C₁-C₄ alkoxy group; and
Z′ is a monovalent functional group represented by Formula 15;

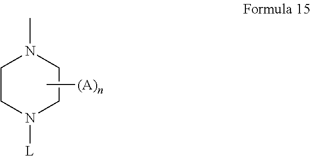

wherein, in Formula 15, n is an integer from 1 to 8;
each A is independently a functional group selected from hydroxy, a C₁-C₄ alkyl group, and a hydroxy C₁-C₄ alkyl group, wherein when n is two or more, two of the two or more A are linked to each other to form an alkylene bridge to form Z′ that is a 7- to 12-membered bridged heterobicycloalkyl ring, or two A are spiro-connected to form a 7- to 12-membered spiroheterocycloalkyl ring; and
L is hydrogen, a C₁-C₄ alkyl, a hydroxy group, or a hydroxyC₁-C₄ alkyl group.

In embodiments, provided herein are methods of treating cancer in a subject in need thereof, comprising administering to the subject a therapeutically effective amount of a compound of Formula (1), or a pharmaceutically acceptable salt thereof, solvate thereof, stereoisomer thereof, tautomer thereof, or combination thereof, wherein the subject has a mutant form of NPM1, DNMT3A, RAS, or a combination thereof:

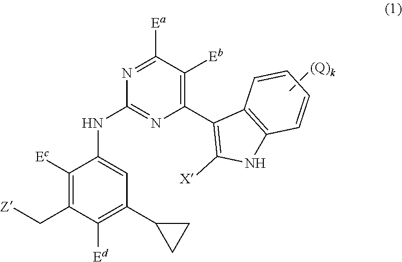

wherein in Formula (1):
Ea is hydrogen, hydroxy or C₁₋₄ alkoxy;
Eb is hydrogen, halogen, C₁₋₄ alkyl or C₁₋₄ fluoroalkyl;
Ec and Ed are each independently hydrogen or hydroxy;
X′ is hydrogen or hydroxy;
k is an integer from 1 to 2;
each Q is independently hydroxy, halogen, C₁₋₄ alkyl, hydroxy C₁₋₄ alkyl or C₁₋₄ alkoxy;
Z′ is a monovalent functional group represented by Formula (2);

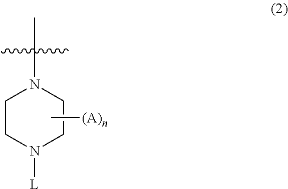

wherein:
each A is independently selected from the group consisting of hydroxy, C₁₋₄ alkyl and hydroxy C₁₋₄ alkyl, wherein at least one A is C₁₋₄ alkyl;
n is an integer from 1 to 2; and
L is hydrogen, C₁₋₄ alkyl, hydroxy or hydroxy C₁₋₄ alkyl.

In embodiments of the methods provided herein, E^{b} is halogen, n is 2, and A is methyl.

In embodiments of the methods provided herein, Z′ is 3,5-dimethylpiperazin-1-yl.

In embodiments of the methods provided herein, E^{b} is chlorine or fluoro.

In embodiments of the methods provided herein, the compound of Formula (1) is a compound of Formula (3), or a pharmaceutically acceptable salt thereof, solvate thereof, stereoisomer thereof, tautomer thereof, or combination thereof;

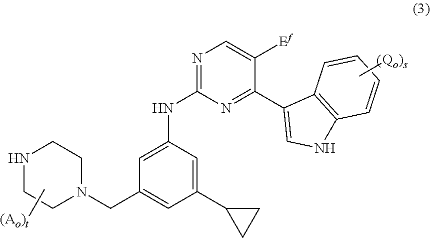

wherein in Formula 3:
Ef is fluorine, chlorine, bromine or iodine;
Qo is hydroxy, halogen, C₁₋₄ alkyl, hydroxy C₁₋₄ alkyl or C₁₋₄ alkoxy;
s is an integer from 1 to 2;
Ao is selected from the group consisting of hydroxy, C₁₋₄ alkyl and hydroxy C₁₋₄ alkyl; and
t is an integer from 1 to 2.

In embodiments of the methods provided herein, the compound of Formula (1) is Compound A:

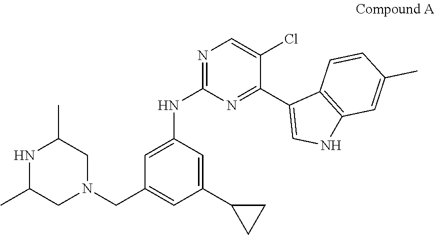

or a pharmaceutically acceptable salt thereof, solvate thereof, stereoisomer thereof, tautomer thereof, or combination thereof.

In embodiments of the methods provided herein, the compound of Formula (1) is Compound I

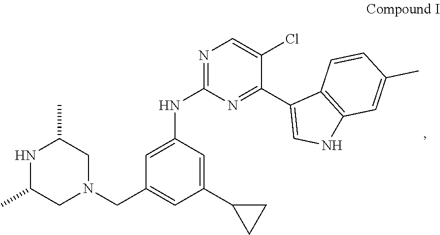

or a pharmaceutically acceptable salt thereof, solvate thereof, stereoisomer thereof, tautomer thereof, or combination thereof.

In embodiments of the methods provided herein, the compound of Formula (1) is selected from the group consisting of:
1) 5-chloro-N-(3-cyclopropyl-5-(((3R,5S)-3,5-dimethylpiperazin-1-yl) methyl) phenyl)-4-(6-fluoro-1H-indol-3-yl) pyrimidin-2-amine;
2) 5-chloro-4-(6-chloro-1H-indol-3-yl)-N-(3-cyclopropyl-5-(((3R,5S)-3,5-dimethylpiperazine-1-yl) methyl) phenyl) pyrimidin-2-amine;
3) 2-((2R,6S)-4-(3-((5-chloro-4-(6-fluoro-1H-indol-3-yl) pyrimidine-2-yl) amino)-5-cyclopropylbenzyl)-2,6-dimethylpiperazine-1-yl) ethan-1-ol;
4) 2-((2R,6S)-4-(3-((5-chloro-4-(1H-indol-3-yl) pyrimidine-2-yl) amino)-5-cyclopropylbenzyl)-2,6-dimethylpiperazine-1-yl) ethan-1-ol;
5) 2-((2R,6S)-4-(3-((5-chloro-4-(6-methyl-1H-indol-3-yl) pyrimidin-2-yl) amino)-5-cyclo propylbenzyl)-2,6-dimethylpiperazin-1-yl) ethan-1-ol;
6) (R)-5-chloro-N-(3-cyclopropyl-5-((3-methylpiperazin-1-yl) methyl) phenyl)-4-(1H-indol-3-yl) pyrimidine-2-amine;
7) (R)-5-chloro-N-(3-cyclopropyl-5-((3-methylpiperazin-1-yl) methyl) phenyl)-4-(6-methyl-1H-indol-3-yl) pyrimidin-2-amine;
8) 5-chloro-N-(3-cyclopropyl-5-(((3R,5S)-3,5-dimethylpiperazin-1-yl) methyl) phenyl)-4-(6-methyl-1H-indol-3-yl) pyrimidin-2-amine;
9) 5-chloro-N-(3-cyclopropyl-5-(((3S,5R)-3-ethyl-5-methylpiperazine-1-yl) methyl) phenyl)-4-(6-methyl-1H-indol-3-yl) pyrimidin-2-amine;
10) 5-chloro-N-(3-cyclopropyl-5-((3,5-dimethylpiperazin-1-yl) methyl) phenyl)-4-(6-methyl-1H-indol-3-yl) pyrimidin-2-amine;
11) N-(3-cyclopropyl-5-(((3R,5S)-3,5-dimethylpiperazin-1-yl) methyl) phenyl)-4-(6-methyl-1H-indol-3-yl) pyrimidin-2-amine;
12) N-(3-cyclopropyl-5-(((3R,5S)-3,5-dimethylpiperazin-1-yl) methyl) phenyl)-5-fluoro-4-(6-methyl-1H-indol-3-yl) pyrimidin-2-amine;
13) N-(3-cyclopropyl-5-(((3R,5S)-3,5-dimethylpiperazin-1-yl) methyl) phenyl)-4-(1H-indol-3-yl)-5-methyl pyrimidin-2-amine;
14) N-(3-cyclopropyl-5-(((3R,5S)-3,5-dimethylpiperazin-1-yl) methyl) phenyl)-5-methyl-4-(6-methyl-1H-indol-3-yl) pyrimidin-2-amine;
15) N-(3-cyclopropyl-5-(((3R,5S)-3,5-dimethylpiperazin-1-yl) methyl) phenyl)-4-(6-methyl-1H-indol-3-yl)-5-(trifluoromethyl) pyrimidin-2-amine;
16) 3-(5-chloro-2-((3-cyclopropyl-5-(((3R,5S)-3,5-dimethylpiperazine-1-yl) methyl) phenyl) amino) pyrimidin-4-yl)-1H-indol-6-yl) methanol;
17) 5-chloro-N-(3-cyclopropyl-5-(((3R,5S)-3,5-dimethylpiperazin-1-yl) methyl) phenyl)-4-(5-methoxy-6-methyl-1H-indol-3-yl) pyrimidin-2-amine;
18) 3-(5-chloro-2-((3-cyclopropyl-5-(((3R,5S)-3,5-dimethylpiperazin-1-yl) methyl) phenyl) amino) pyrimidin-4-yl)-6-methyl-1H-indol-5-ol;
19) 3-(5-chloro-2-((3-cyclopropyl-5-(((3R,5S)-3,5-dimethylpiperazin-1-yl) methyl) phenyl) amino) pyrimidin-4-yl)-6-methylindolin-2-on;
20) 5-chloro-N-(3-cyclopropyl-5-(((3R,5S)-3,5-dimethylpiperazin-1-yl) methyl) phenyl)-4-methoxy-6-(6-methyl-1H-indol-3-yl) pyrimidin-2-amine;
21) 5-chloro-2-((3-cyclopropyl-5-(((3R,5S)-3,5-dimethylpiperazin-1-yl) methyl) phenyl) amino)-6-(6-methyl-1H-indol-3-yl) pyrimidin-4-ol;
22) 3-(5-chloro-2-((3-cyclopropyl-5-(((3R,5S)-3,5-dimethylpiperazin-1-yl) methyl) phenyl) amino) pyrimidin-4-yl)-6-methyl-1H-indol-7-ol;
23) 2-((5-chloro-4-(6-methyl-1H-indol-3-yl) pyrimidin-2-yl) amino)-4-cyclopropyl-6-(((3R,5S)-3,5-dimethylpiperazin-1-yl) methyl) phenol;
24) 4-((5-chloro-4-(6-methyl-1H-indol-3-yl) pyrimidin-2-yl) amino)-2-cyclopropy-6-(((3R,5S)-3,5-dimethylpiperazin-1-yl) methyl) phenol;
25) (R)-5-chloro-N-(3-cyclopropyl-5-((3,3,5-trimethylpiperazin-1-yl) methyl) phenyl)-4-(6-methyl-1H-indol-3-yl) pyrimidin-2-amine;
26) ((2R,6R)-4-(3-((5-chloro-4-(6-methyl-1H-indol-3-yl) pyrimidin-2-yl) amino)-5-cyclopropylbenzyl)-6-methylpiperazin-2-yl) methanol;
27) (R)-5-chloro-N-(3-cyclopropyl-5-((5-methyl-4,7-diazaspiro [2.5] oxtan-7-yl) methyl) phenyl)-4-(6-methyl-1H-indol-3-yl) pyrimidin-2-amine;
28) 5-chloro-N-(3-cyclopropyl-5-(((3R,5R)-3,5-dimethylpiperazin-1-yl) methyl) phenyl)-4-(6-methyl-1H-indol-3-yl) pyrimidin-2-amine;
29) 5-chloro-N-(3-cyclopropyl-5-(((3S,5S)-3,5-dimethylpiperazin-1-yl) methyl) phenyl)-4-(6-methyl-1H-indol-3-yl) pyrimidin-2-amine;
30) 5-chloro-N-(3-cyclopropyl-5-(((3R,5S)-3,4,5-trimethylpiperazin-1-yl) methyl) phenyl)-4-(6-methyl-1H-indol-3-yl) pyrimidin-2-amine;
31) (2R,6S)-4-(3-((5-chloro-4-(6-methyl-1H-indol-3-yl) pyrimidin-2-yl) amino)-5-cyclopropylbenzyl)-2,6-dimethylpiperazin-1-ol; and
32) (2R,6S)-4-(3-cyclopropyl-5-((4-(6-methyl-1H-indol-3-yl) pyrimidine-2-yl) amino) benzyl)-2,6-dimethylpiperazin-1-ol.

In embodiments of the methods provided herein, the cancer is a hematological cancer.

In embodiments of the methods provided herein, the hematological cancer is leukemia.

In embodiments of the methods provided herein, the leukemia is acute myelogenous leukemia (AML), chronic myelogenous leukemia (CMIL), acute lymphocytic leukemia (ALL), chronic lymphocytic leukemia, CLL), acute promyelocytic leukemia (APL), hairy cell leukemia, chronic neutrophilic leukemia (CNL), and the like.

In embodiments of the methods provided herein, the leukemia is acute myeloid leukemia, acute lymphocytic leukemia, or chronic myelogenous leukemia.

In embodiments of the methods provided herein, the leukemia is acute myeloid leukemia (AML).

In embodiments of the methods provided herein, the acute myeloid leukemia (AML) herein is acute myeloid leukemia with a FLT3 mutation. In one embodiment, the acute myeloid leukemia comprises a mutant FLT3 polynucleotide-positive myeloid leukemia, a ITD positive acute myeloid leukemia in the FLT3 gene, or an acute myeloid leukemia having a FLT3 point mutation.

In embodiments of the methods provided herein, the AML is relapsed or treatment-refractory (R/R) AML.

In embodiments of the methods provided herein, the AML is relapsed or treatment-refractory (R/R) AML with MDS-related changes.

In embodiments of the methods provided herein, the subject has failed prior therapy with chemotherapy, hematopoietic stem cell transplantation (HSCT) therapy and/or therapy with other FLT3 inhibitors.

In embodiments of the methods provided herein, the subject has failed prior therapy with chemotherapy.

In embodiments of the methods provided herein, the subject has failed prior therapy with hematopoietic stem cell transplantation (HSCT) therapy.

In embodiments of the methods provided herein, the subject has failed prior therapy with therapy with other FLT3 inhibitors.

In embodiments of the methods provided herein, the subject has failed prior therapy with a DNA hypomethylating agent (HMA) and other FLT3 inhibitors.

In embodiments of the methods provided herein, the subject has failed prior therapy with one or more chemotherapeutic agents and other FLT3 inhibitors.

In embodiments of the methods provided herein, the subject has received from 1 to 8 prior cancer (e.g., AML) therapies, including 1, 2, 3, 4, 5, 6, 7, or 8 prior therapies. In embodiments, the subject has received at least one prior cancer (e.g., AML) therapy. In embodiments, the subject has received at least two prior cancer (e.g., AML) therapies.

In embodiments of the methods provided herein, the subject has received prior therapy with a DNA hypomethylating agent (HMA). In embodiments, the subject has failed prior therapy with an HMA. Hypomethylating agents include for example, azacitidine, decitabine, and combinations thereof. In embodiments, the subject has failed prior therapy with azacitidine, decitabine, or a combination thereof.

In embodiments of the methods provided herein, the subject has received prior therapy with a chemotherapeutic agent. In embodiments, the subject has failed prior therapy with a chemotherapeutic agent. Chemotherapeutic agents include cytarabine, daunorubicin, idarubicin, doxorubicin, fludarabine and the like. In embodiments, the chemotherapeutic agent is cytarabine, daunorubicin, fludarabine, or combinations thereof. In embodiments the chemotherapeutic agent is cytarabine, daunorubicin, or a combination thereof. In embodiments the chemotherapeutic agent is cytarabine, fludarabine, or a combination thereof.

In embodiments of the methods provided herein, the subject has received prior therapy with a Bcl-2 inhibitor such as venetoclax. In embodiments, the subject has failed prior therapy with a Bcl-2 inhibitor.

In embodiments of the methods provided herein, the subject has received prior therapy with a FLT-3 inhibitor, including but not limited to midostaurin, gilteritinib and combinations thereof. In embodiments the subject has failed prior therapy with a FLT-3 inhibitor, including but not limited to midostaurin, gilteritinib and combinations thereof. In embodiments, the patient has received prior therapy with midostaurin. In embodiments, the patient has received prior therapy with gilteritinib. In embodiments, the patient has received prior therapy with midostaurin and gilteritinib.

In embodiments of the methods provided herein, the subject has received prior HSCT therapy.

In embodiments of the methods provided herein, the subject has a mutant form of FLT3 and a mutant form of NPM1, DNMT3A, RAS, or a combination thereof, or a mutant form of RAS:

In embodiments of the methods provided herein, the subject has a mutant form of NPM1, DNMT3A, and/or RAS.

In embodiments of the methods provided herein, the subject has a mutant form of FLT3, a mutant form of NPM1, a mutant form of DNMT3A and a mutant form of RAS.

In embodiments of the methods provided herein, the subject has a mutant form of NRAS.

In embodiments of the methods provided herein, the subject has a mutant form of KRAS.

In embodiments of the methods provided herein, the subject has a mutant form of FLT3 and a mutant form of NPM1.

In embodiments of the methods provided herein, the subject has a mutant form of FLT3, a mutant form of NPM1 and a mutant form of DNMT3A.

In embodiments of the methods provided herein, the subject has a mutant form of RAS, e.g., a mutant form of NRAS or a mutant form of KRAS.

In embodiments of the methods provided herein, the subject has a mutant form of RAS and a wild type FLT3.

In embodiments of the methods provided herein, the subject has a mutant form of RAS and a mutant form of FLT3.

In embodiments of the methods provided herein, the mutated FLT3 comprises an internal tandem duplication (ITD) mutation and/or at least one FLT3 point mutation.

In embodiments of the methods provided herein, the FLT3 point mutation is on one or more residues selected from the group consisting of D835, F691, K663, Y842 and N841.

In embodiments, the FLT3 mutation may be a FLT3 mutation in the internal tandem duplication (ITD) or one or more activating point mutations such as D835Y, D835V, I836.

In embodiments of the methods provided herein, the FLT3 point mutation is F691L mutation.

In embodiments of the methods provided herein, the FLT3 point mutation is ITD-F691L double mutation.

In embodiments of the methods provided herein, the mutated FLT3 comprises at least one mutation in the tyrosine kinase domain of FLT3.

The mutation of FLT3-TKD may include one or more amino acid mutations in the positional region 823 to 861 of the FLT3 amino acid sequence. The mutation of the TKD may include a mutation of at least one amino acid selected from the group consisting of numbers 835, 836, and 842 of the FLT3 amino acid sequence. For example, the mutation of the TKD may include a mutation of amino acid 835 in the FLT3 amino acid sequence. For example, the mutation in TKD may be one in which the aspartic acid No. 835 of the FLT3 amino acid sequence is substituted with valine, tyrosine, histidine, glutamic acid, or asparagine. For example, the mutation of TKD may be one in which isoleucine 836 of the FLT3 amino acid sequence is substituted with leucine or aspartic acid. As another example, the mutation of TKD may be one in which tyrosine 842 of the FLT3 amino acid sequence is substituted with cysteine or histidine. Also, the mutation may be FLT3 (D835Y).

The FLT3-TKD mutation may be a mutation in at least one amino acid selected from the group consisting of 621, 627, 676, 691, and 697 of the FLT3 amino acid sequence. For example, the mutation of TKD may be one in which phenylalanine at position 691 of the FLT3 amino acid sequence is substituted with leucine. For example, the mutation may be FLT3 (F691L).

The mutation of the TKD may be one that further comprises an internal tandem duplication (ITD). For example, the mutation may be FLT3 (ITD/D835Y) or FLT3 (ITD/F691L).

In embodiments, the FLT3-TKD mutation may include any one selected from FLT3 (D835Y), FLT3 (F691L), FLT3 (F691L/D835Y), FLT3 (ITD/D835Y), FLT3 (ITD/F691L), and combinations thereof.

In embodiments of the methods provided herein, the mutated FLT3 comprises an internal tandem duplication (ITD) mutation.

In embodiments of the methods provided herein, the mutated FLT3 comprises at least one point mutation is in the activation loop of FLT3.

In embodiments of the methods provided herein, the subject comprises one or mutations selected from the group consisting of NPM1-DNMT3A-FLT3-ITD, NRAS-ITD, KRAS-NPM1-DNMT3A-FLT3-ITD, NRAS-FLT3-ITD, NRAS-FLT3-WT, NPM1-FLT3-ITD.

FLT3 is a member of the class III receptor tyrosine kinase (TK) family that is commonly expressed on the surface of hematopoietic stem cells. FLT3 and its ligands play important roles in proliferation, survival and differentiation of pluripotent stem cells. FLT3 is expressed in many AML cases. In addition, activated FLT3 with intragenic tandem duplication (ITD) in and around the proximal domain and tyrosine kinase domain (TKD) mutations near D835 in the activation loop are 28% to 34% and 11% to 11% of AML cases, respectively. present at 14%. These activating mutations in FLT3 are tumorigenic and exhibit transforming activity in cells. Patients with FLT3-ITD mutations have a poorer prognosis in clinical studies, higher relapse rates, shorter duration of remission from initial treatment (6 months versus 11.5 months in patients without FLT3-ITD mutations), and lower disease-free survival rates. reduced and the OS is reduced. The incidence of recurrence after hematopoietic stem cell transplantation (HSCT) is also higher for patients with FLT3-ITD. Similar to the prognosis for first-line treatment, patients with relapsed/refractory FLT3-positive AML have a lower rate of remission with rescue chemotherapy, a shorter remission period to second-line relapse, and compared to FLT3-negative patients with salvage chemotherapy. It has a reduced OS.

In embodiments, the methods of the present disclosure demonstrate efficacy in treating acute myeloid leukemia having an FMS-like tyrosine kinase 3 (FLT3) mutation, which leads to a high risk of recurrence after treatment, a poor prognosis, and a decrease in overall survival rate.

In embodiments, the methods of the present disclosure provide clinical benefits even in patients with acute myeloid leukemia who are resistant to conventional therapeutic agents.

In embodiments, the methods provided herein can overcome the resistance of acute myeloid leukemia (AML) treatment.

In embodiments of the methods provided herein, the compound of the present disclosure (e.g., a compound of Formula (1), Formula (3), Formula (14), Compound A or Compound I) is formulated as a pharmaceutical formulation further comprising a pharmaceutically acceptable excipient.

As an active ingredient included in the pharmaceutical composition, the compound of the present disclosure (e.g., a compound of Formula (1), Formula (3), Formula (14), Compound A or Compound I) is provided in an effective amount for treatment or prevention of an individual or patient, and may be administered orally or parenterally as desired, and when administered orally, the active ingredient For parenteral administration, 1 body weight per day based on the active ingredient so as to be administered in an amount of, for example, 0.01 to 1000 mg, 0.01 to 500 mg, 0.1 to 300 mg, or 0.1 to 100 mg per 1 kg of body weight per day as a standard For example, it may be included to be administered in an amount of 0.01 to 100 mg or 0.1 to 50 mg per kg, and the composition may be administered in 1 to several divided doses. The dose to be administered to a specific individual or patient should be determined in light of several related factors such as the patient's weight, age, sex, health condition, diet, administration time, administration method, and disease severity, and can be appropriately adjusted or decreased by a specialist. It should be understood that the above dosage is not intended to limit the scope of the present invention in any way.

In embodiments of the methods provided herein, the dosage, the frequency of administration, or the administration method of the compound used in the treatment method may vary depending on the subject to be treated, the severity of the disease or condition, the rate of administration, and the judgment of the prescribing physician. Usually, the dosage for a person weighing 70 kg may be administered in an amount of 0.1 to 2,000 mg, for example, 1 to 1,000 mg or 10 to 2,000 mg per day. The frequency of administration may be 1 to several times, for example, 1 to 4 times, or an on/off schedule may be administered, and the administration method may be administered through an oral or parenteral route. In some cases, dosages lower than the aforementioned ranges may be more suitable, higher dosages may be used without producing deleterious side effects, and higher dosages may be divided into several smaller dosages throughout the day. can be A physician having ordinary skill in the related art can easily determine and prescribe the dosage of the compound to be used as needed. For example, a physician may start a dose of a compound of the present invention used in a pharmaceutical composition at a level lower than that required to achieve the desired therapeutic effect, and gradually increase the dosage until the desired effect is achieved.

Treatment drugs according to one specific example may be administered at effective treatment intervals. The period of time or cycle of administration may 1 week, 28 days, 1 month, 2 months, 3 months, or 4 months, or more in total. The treatment drug may be administered daily for the entire duration or only a portion of a period or cycle.

In embodiments, the treatment drug (e.g., a compound of Formula (1), Formula (3), Formula (14), Compound A or Compound I) is administered as an oral once-daily dose in a 28-day cycle.

In another embodiment, the dosage amount of the treatment drug (e.g., a compound of Formula (1), Formula (3), Formula (14), Compound A or Compound I) ranges between about 10 mg to about 300 mg. In another embodiment, the dosage amount ranges between about 20 mg to about 240 mg. In another embodiment, the dosage amount ranges in between about 40 mg and about 200 mg. In another embodiment, the dosage amount ranges between about 80 mg, and about 160 mg, or any ranges or subranges therein or in between.

In a specific embodiment, the dosage amount is about 20 mg, 30 mg, 40 mg, 50 mg, 60 mg, 70 mg, 80 mg, 90 mg, 100 mg, 120 mg, 140 mg, 160 mg, 180 mg, 200 mg, 220 mg, 240 mg, 260 mg, 280 mg, and 300 mg.

In a specific embodiment, the dosage amounts are administered to a patient once a day, twice a day, three times a day, or four times a day. In a specific embodiment, the dosage amounts are administered to a patient once a day. In a specific embodiment, the dosage amounts are administered to a patient twice a day. In a specific embodiment, the dosage amounts are administered to a patient three times a day. In a specific embodiment, the dosage amounts are administered to a patient four times a day.

In another embodiment, the dosing will be administered in one week cycles, 2 week cycles, three week cycles, 4 week cycles, 5 week cycles, 6 week cycles, 7 week cycles or 8 week cycles.

In embodiments of the methods provided herein, Compound I is administered in an amount of about 10 mg to about 300 mg.

In embodiments of the methods provided herein, Compound I is administered in an amount of about 40 mg to about 160 mg.

In embodiments of the methods provided herein, Compound I is administered in an amount of about 80 mg, about 120 mg, and/or about 160 mg.

In embodiments of the methods provided herein, routes of administration include, but are not limited to, oral, intravenous, intraarterial, intraperitoneal, intradermal, transdermal, intrathecal, intramuscular, intranasal, transmucosal, subcutaneous and rectal administration.

In a specific embodiment, the methods may include treating cancer in a subject in need thereof, comprising administering to the subject a therapeutically effective amount of Compound A or Compound I, or a pharmaceutically acceptable salt thereof, solvate thereof, stereoisomer thereof, tautomer thereof, or combination thereof, wherein the cancer is relapsed or treatment-refractory (R/R) acute myeloid leukemia (AML). In a specific embodiment, the subject has wildtype or a FLT3 mutation. In another specific embodiment, the FLT3 mutation is a ITD or TKD mutation. In another specific embodiment, the mutated FLT3 comprises an internal tandem duplication (ITD) mutation and/or at least one FLT3 point mutation. In another specific embodiment, the at least one point mutation is on one or more residues selected from the group consisting of D835, F691, K663, Y842 and N841. In another specific embodiment, the mutated FLT3 comprises at least one mutation in the tyrosine kinase domain of FLT3. In another embodiment, the mutated FLT3 comprises an internal tandem duplication (ITD) mutation. In another embodiment, the mutated FLT3 comprises at least one point mutation is in the activation loop of FLT3.

In another embodiment, the subject had prior BCL-2 inhibitor therapy or FLT3 inhibitor therapy. In another embodiment, the BCL-2 inhibitor is venetoclax. In another embodiment, the FLT3 inhibitor is lestaurtinib, sorafenib, midostaurin, quizartinib, crenolanib, and/or gilteritinib.

In another embodiment, the dose of Compound I or compound A is between about 20 mg to about 250 mg. In a specific embodiment, the dose is administered orally to the subject once daily. In another specific embodiment, the dose of Compound I is about 40 mg, about 80 mg, about 120 mg, about 160 mg, or about 200 mg. In another embodiment, the dosing is administered once daily for 28 days.

In a specific embodiment, the 28 days of administration is a cycle that is repeated more than once, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times or more than 20 times to the subject.

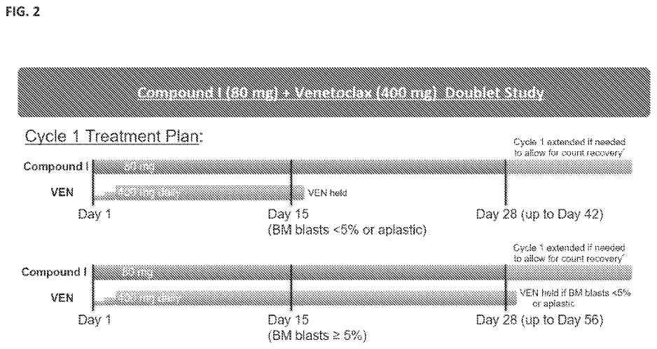

In another specific embodiment, Compound A or Compound I is administered in combination with a BCL-2 inhibitor. In a specific embodiment, the BCL-2 inhibitor is venetoclax. In a specific embodiment, the combination treatment is administered by the methods described above. In another embodiment, the combination treatment schedule is as described in FIG. 2. In another embodiment, Compound I or Compound A is administered once daily and the BCL-2 inhibitor or venetoclax is administered once daily. In another embodiment, Compound I is administered at about 80 mg. In another specific embodiment, venetoclax is administered at a daily dose of about 100 mg to about 500 mg. In another specific embodiment, venetoclax is administered at a daily dose of about 400 mg. In another specific embodiment, venetoclax is administered at a lower daily dose at day 1 or day 2 of a treatment plan, with an increased daily dose at day 3, day 4, day 5, day 6, day 7, day 8, day 9 or day 10.

In embodiments of the methods provided herein, formulations for administration may be formulated and used in any suitable form according to conventional methods, including oral dosage forms such as tablets, powders, granules, capsules, suspensions, emulsions, syrups, aerosols, etc., external preparations such as ointments and creams, injections, and suppositories and sterile injection solutions, etc.

In embodiments of the methods provided herein, the compounds of the present disclosure (e.g., a compound of Formula (1), Formula (3), Formula (14), Compound A or Compound I) is administered orally or parenterally.

### EXAMPLES

### Example 1: Clinical Study of Compound I in Patients with AML and Evaluation of the Efficacy of Compound I in AML Patients with Adverse Mutations

This study is an open-label, first-in-human, dose escalation, exploration, and expansion study of Compound I as a single agent in patients with relapsed or refractory (“R/R”) AML. Cycle 1 in dose escalation is defined as 30 days, all other cycles lasting at least 28 days. Patients will receive oral Compound I at QD on a continuous basis, with the exception of Cycle 1 Day 2 in Part A. The study treatment can continue until a discontinuation criterion is met.

An objective of this study was to explore the potential of Compound I to treat AML patients with adverse mutations including but not limited to FLT-3, NPM1, RAS, and DNMT3A.

### Study Design

The initial dose level of Compound I as a single agent was 20 mg daily and the decision to dose escalate to the next dose level is made based on the assessment of safety variables including moderate toxicity (MT, a Grade 2 AE judged by the investigator to be related to study drug (except for hematologic toxicities)) or dose limiting toxicity (DLT). This study includes 3 parts: Part A: Dose Escalation; Part B: Dose Exploration; and Part C: Dose Expansion.

### Part A: Dose Escalation:

Part A includes the initial dose escalation cohorts. Provisional dose escalation scheme with the planned doses of Compound I are as follows: 20 mg, 30 mg, 40 mg, 60 mg, 80 mg, 120 mg, 160 mg, 200 mg, or 240 mg. Patients are treated daily in 28-day cycles except for the Cycle 1 (30 days). The DLT observation period is during Cycle 1 starting with the first dose on Day 1. Patients in Cycle 1 have PK sampling performed after receiving a single dose of the study drug on Day 1. This study follows an accelerated titration design. Dose levels are set at around 50% increments. One patient is treated at the starting dose level of 20 mg. If no DLT or MT is identified, the next patient is enrolled at double the dose level, i.e., dose level 3 (40 mg). This dose escalation approach will continue until the first instance of a DLT or MT (a Grade 2 AE judged by the investigator to be related to study drug (except for hematologic toxicities) occur.

After DLT evaluable patients are identified or MT is observed in each dose level in the accelerated titration design, data pertaining to dose escalation decisions are reviewed. Available data including demographics, AEs, laboratory assessments, dose administration, and any other pertinent information relevant to patient's safety will be reviewed. The determination will be made as to whether dose escalation should continue and, if so, at what dose level and schedule. This data review may be performed at any time. In addition, if safety assessment is required a data review may take place ad-hoc.

When a DLT or MT is observed in a patient, the dose escalation schedule will stop the double-dose level method and follow the next consecutive dose level in utilizing the modified 3+3 design. Modified 3+3 design testing each consecutive dose level may also be followed if recommended through the safety review meeting (RM) based on the review of available PK data.

After dose escalation design converting to 3+3 design, 3 patients are treated at each dose level. If no DLTs are observed among 3 patients, the subsequent patients are treated at the next dose level. If one DLT is observed among 3 patients in a dose level (1/3 DLTs observed), 3 more patients are enrolled at that dose level. If the 3 additional patients do not experience a DLT (≤1/6 DLTs observed), the next dose level is initiated. If 2 or more DLTs occur in a dose level (≥2/3 or ≥2/6 DLTs observed), the dose will be considered intolerable and dose escalation will be stopped. MTD will be determined at the next lower level or, if appropriate, a dose between the highest tolerable one and the intolerable one will be further explored to determine MTD.

### Part B: Dose Exploration:

Part B is the dose exploration cohort. Patients will be treated daily in 28-day cycles. The DLT observation period is during Cycle 1 starting with the first dose on Day 1. At any dose level, if no DLTs are observed in the initial 3 patients (0/3 DLTs observed) in dose escalation cohort (Part A), the dose level will be expanded to enroll up to 6 patients (including initial 3 patients) and DLT assessment will be performed. If one or fewer DLTs are observed in the 6 patients (≤1/6 DLTs observed), the dose level continues to enroll patients up to 20 patients. If 2 or more DLTs occur among 6 patients (≥2/6 DLTs observed) in a dose level, the additional enrollment will be stopped. If one DLT is observed in the 6 patients (1/6 DLT observed) in the dose escalation cohort (Part A), up to 20 patients can be enrolled in the dose exploration cohort (Part B) at the dose level. The planned doses of Compound I are for Part B are also as follows: 20 mg, 30 mg, 40 mg, 60 mg, 80 mg, 120 mg, 160 mg, 200 mg, or 240 mg.

If both Part A and Part B are open concurrently, newly enrolled patients are assigned to Part A first. If multiple dose levels are simultaneously explored, patient enrollment is carried out preferentially at the lowest dose of the explored dose levels. As several complete responses are observed at 80 mg dose, the 40 mg dose level may be expanded up to a total of 20 patients to further explore the safety, PK, and activity at this dose level. Part B can occur concurrently with dose expansion in Part C.

At least half of the patients in Part B dose level have AML with FLT3 mutation (e.g., ITD or activating point mutations such as D835Y, D835V, I836) including patients in Part A. If 10 FLT3-unmutated patients are enrolled at a dose level, that level will be closed to further enrollment of FLT3-unmutated patients. Patients with or without a documented FLT3 mutation will be enrolled and samples will be collected at screening visit to confirm or evaluate FLT3 mutation status. If the FLT3 mutation status at the time of enrollment is unknown, patients will be considered to have the FLT3 mutation status determined by their most recent prior genetic test. FLT3-ITD or TKD mutation will be determined with FDA-approved test or validated assay in central laboratory.

The safety in the dose exploration cohort (Part B) will be monitored using Bayesian logistic regression modeling, based on the DLT rate observed in patients from both Part A and Part B.

If at least one patient in Part A at any dose level achieves clinical response (CR), CR with partial hematological recovery (CRh), CR with incomplete platelet recovery (CRp), CR with incomplete hematological recovery (CRi), or partial remission (PR)), then the dose level can continue to enroll a minimum of 3 patients in either Part A or Part B. In parallel with the accelerated dose escalation, DLT assessment will be performed for the initial 3 patients at the dose level of exploration part for safety monitoring in the same scheme as in 3+3 design.

If no DLTs are observed in the initial 3 patients or the 3 following patients do not experience a DLT after one patient in the initial 3 patients experiences a DLT (0/3 or 1/6 DLTs observed), the dose level will continue to enroll additional patients. Additionally, if fewer than 2 responses (composite CR [CR+CRh+CRi+CRp](CRc)+PR) are achieved in 12 patients who complete 2 treatment cycles, the dose level will stop further enrollment. Otherwise, the dose level continues to enroll patients up to 20 evaluable patients. For the patients in Part B, Bayesian logistic regression model will be also applied as supportive analysis for safety assessment.

### Part C: Dose Expansion:

Part C is the dose expansion cohort to determine safety and tolerability of Compound I as a single agent, or the Compound I plus venetoclax combination treatment group. Within each group, approximately half of patients have FLT3 mutations, and the other half are FLT3-unmutated. Among the FLT3-mutated patients in the Compound I single agent treatment group, at least 16 (evaluable) patients have received prior treatment with a FLT3 inhibitor. Of the FLT3-unmutated patients in the Compound I single agent treatment group, at least 12 (evaluable) patients have a TP53 mutation or complex karyotype. The initial single agent dose of Compound I is 120 mg daily, and the starting dose in the Compound I plus venetoclax treatment group is 80 mg daily. In additional dosing studies, the dosing is 20 mg to 200 mg once daily of Compound I. Patients are assigned to a treatment group based on the number of slots available. Treatment will be in 28-day cycles, and patients receive daily dosing with Compound I in all treatment groups. Patients not evaluable for response assessment may be replaced. For the single agent group, after 6 patients have completed Cycle 1, an SRM will be held to complete a safety review that includes all subjects in the study to date. Recommendations of dosage adjustments based on the availability of safety information may be made at the SRM.

Response assessments will be performed on Cycle 1 Day 15 and at subsequent points depending on response. Events that occur in Part C that would be considered a DLT if occurred in Part A or Part B will be reviewed and assessed at the Safety Review Meetings (SRM). For the combination treatment group, a safety review meeting will be held after each set of 3 patients complete Cycle 1. The SRM can recommend changes to the dosing schedule for subsequent patients.

In both Part A and Part B, a patient that receives less than 80% of the intended dose during Cycle 1 (e.g., misses 6 daily doses or leaves the study for reasons other than a DLT) will not be evaluable for DLT and will be replaced by another patient in the dose level. In addition, if after enrollment any patient is found not to fulfill any inclusion/exclusion criteria that would adversely affect safety or efficacy evaluation of that patient, they may be replaced after discussion between the Principal Investigator and Medical Monitor. Based on evaluation of the primary objective, additional cohorts with potentially modified target patients and regimen of study drug may be accrued.

For Part C, patients may be replaced if they are deemed not evaluable for response by Cycle 3 Day 1 (have not received 2 cycles of treatment or have discontinued for a reason other than disease progression).

Provisional dose escalation scheme with the planned doses for Part A is presented. However, this plan may change based on the recommendations from the Cohort Review Meeting.

DLT assessment will be determined. A DLT is defined as any of the following events that occur within Cycle 1 starting with the first dose taken on Day 1 for both Part A and Part B, and that is considered to be related to study drug.

Any Grade ≥3 non-hematologic or extramedullary toxicity.

The following exceptions are noted:
a. Alopecia, anorexia, or fatigue of any grade.
b. Grade 3 nausea or vomiting or diarrhea if not requiring hospitalization, TPN, or tube feeding.
c. Grade 3 fever with neutropenia, with or without infection.
d. Grade 3 infection.
e. Hematologic toxicity will not be considered as a DLT.

However, prolonged myelosuppression defined as ANC<500 for more than 21 days off therapy in the absence of evidence of active leukemia in the marrow or blood will be considered as a DLT.

Any Grade 4 organ toxicity is a DLT.

Monitoring for excess toxicity is to continue to be performed in Part C. All available safety, tolerability, and PK data is reviewed for patients in the combination treatment group to determine if any adjustments need to be made to the venetoclax and/or Compound I doses for subsequent patients.

### FLT3 Mutation Status—Bone Marrow or Peripheral Blood

A central FLT3 mutation test will be performed for Part A, Part B, and Part C. Bone marrow samples will be collected in a heparin tube and used to determine or confirm FLT3 mutation status. Bone marrow should be collected by aspiration during the Screening period. Approximately 1 mL should be collected in a heparinized tube. If bone marrow sample is unobtainable (e.g., dry tap), 3 mL of the peripheral blood sample should be taken instead at the Screening visit. Presence of FLT3 ITD or TKD mutation in collected samples will be analyzed by FDA-approved test or FDA-validated assay in central laboratory. Samples collected at screening visit could be used for development of diagnostic assay.

### Resistant Mutation—Bone Marrow or Blood Peripheral Blood

A bone marrow sample will be collected in ethylenediaminetetraacetic acid (EDTA) tube. Bone marrow should be collected by aspiration during the screening period and at the EOT visit. Approximately 1 mL should be taken in an EDTA tube. If bone marrow sample is unobtainable (e.g., dry tap) or has not enough volume, 3 mL of the peripheral blood sample should be taken in EDTA tube instead.

Mutation status of FLT3 and other genes not limited to NRAS and Kirsten RAS, that could affect efficacy of Compound I will be investigated with collected samples. Resistance mutations to Compound I treatment will be elucidated with this analysis. Clinical Laboratory Improvement Amendments (CLIA) certified or validated next generation sequencing (NGS) assay will be used for analysis and these assays are used for exploratory purposes only.

### Results

As shown in Table 1 and Table 2 below a clinical response was achieved in patients with adverse mutations. Responses across a spectrum of genetically-defined populations with highly adverse mutations were observed. Most responders bridged to potentially life-saving transplant.

TABLE 1
Clinical Responses in R/R AML Patients with Adverse
Mutations Following Treatment with Compound I

| Patient | | | Dose | | Best | Bridged to |
|---|---|---|---|---|---|---|
| No | Mutations | FLT3 Status | Level | | Response | HSCT |
| 1 | IDH2 SRSF2 | WT | 80 | mg | CR | Yes |
| 2 | TP53 | WT | 80 | mg | CR | No |
| 3 | NPM1 DNMT3A | ITD | 80 | mg | CR | Yes |
| 4 | NRAS RUNX1 | ITD | 80 | mg | CRh | Yes |
| 5 | RUNX1 SF3B1 RB1 | TKD - Prior | 80 | mg | CR | Yes |
| | | FLT3i | | | | |
| 6 | KRAS NPM1 DNMT3A | ITD - Prior | 120 | mg | PR | No |
| | PTPN11 | FLT3i | | | | |
| 7 | MLL-PTD RUNX1 | ITD -Prior | 120 | mg | CRi | Yes |
| | | FLT3i | | | | |
| 8 | Not Reported | ITD | 120 | mg | PR | No |
| 9 | NRAS BCOR U2AF1 | WT | 160 | mg | CR | Tx Ongoing |
| | SETBP1 | | | | | |
| 10 | NPM1 | ITD | 160 | mg | CRp | Tx Ongoing |
| 11 | NPM1 IDH1 DNMT3A | ITD | 160 | mg | PR | No |
| 12 | ASXL1 CBL | WT | 80 | mg | PR | No |
| 13 | Not Reported | ITD | 160 | mg | SD | Tx Ongoing |
| 14 | Not Reported | WT | 40 | mg | CRi | Tx Ongoing |

TABLE 2
Response Rates in Efficacy Evaluable R/R AML
Patients from Active/Safe Doses (40, 80, 120, 160 mg)
Compound I Best Response in Each r/r AML Population

| Population | Dose Level(s) | Overall Response Rate | |
|---|---|---|---|
| FLT3+ | 80 mg | 42.9% | (3/7) |
| FLT3+ | 120 mg | 42.9% | (3/7) |
| FLT3+ | Across 80 mg, 120 mg, | 38.1% | (8/21) |
| | 160 mg | | |
| FLT3+ \| NPM1+ | Across 80 mg, 120 mg, | 66.7% | (4/6) |
| | 160 mg | | |
| FLT3+ \| NPM1+ \| | Across 80 mg, 120 mg, | 75% | (3/4) |
| DNMT3A+ | 160 mg | | |
| FLT3+ \| Prior FLT3i | 80 mg | 33.3% | (1/3) |
| FLT3+ \| Prior FLT3i | 120 mg | 40% | (2/5) |
| N/K-RAS+ (FLT3+/−) | Across 80 mg, 120 mg, | 37.5% | (3/8) |
| | 160 mg | | |
| FLT3-WT | Across 80 mg, 120 mg, | 19.0% | (4/21) |

| | 160 mg | | |
|---|---|---|---|
| Population | Dose Level(s) | CRc Response Rate | |
| FLT3+ | Across 80 mg, 120 mg, | 23.8% | (5/21) |
| | 160 mg | | |
| FLT3+ \| NPM1+ | 160 mg | 50.0% | (1/2) |
| FLT3+ \| NPM1+ | Across 80 mg, 120 mg, | 33.3% | (2/6) |
| | 160 mg | | |
| FLT3-WT \| TP53+ | Across 80 mg, 120 mg, | 33.3% | (1/3) |
| | 160 mg | | |
| FLT3+ | 80 mg | 42.9% | (3/7) |
| NRAS+ | Across 80 mg, 120 mg, | 33.3% | (2/6) |

160 mg

### Example 2: Study and Evaluation of the Efficacy of Compound I in R/R AML Patients

A study was further performed/continued using the protocol as described above in Example 1. Specially, Compound I as monotherapy, or in combination with venetoclax, was tested on relapsed or refractory (R/R) AML human patients. Such patients can include, but are not limited to venetoclax-naïve patients, prior venetoclax patients, FLT-3 WT and FLT-3 mutated AML patients and prior FLT3 inhibitor patients. The studies showed that Compound I as a single agent is well tolerated and particularly active in VEN-naïve relapsed or refractory AML. The studies also show that Compound I and venetoclax as a combination is well tolerated in broad populations of R/R AML, including FLT-3 mutant and FLT-3 wildtype (WT) AML patients, including patients with prior FLT3 inhibitors. Table 3 below shows for example the composite complete remission response rate of R/R AML patients from a therapeutic dose of 80-160 mg QD of Compound I as a single agent using the protocol as described in Example 1.

TABLE 3
Compound I Response Rates in R/R AML
Patients from Therapeutic Doses (80-160 mg QD)
Compound I Composite Complete Remission (CRc)

| | |
|---|---|
| Subgroups | CRc Response Rate (n = 68) |

| Overall | 13% | (9/68) |
|---|---|---|
| Venetoclax Naïve patients | 29% | (8/28) |
| Prior Venetoclax patients | 3% | (1/40) |
| FLT3-Mutated Patients | 18% | (5/28) |
| Venetoclax Naïve patients | 42% | (5/12) |
| Prior Venetoclax patients | 0% | (0/16) |
| Prior FLT3 inhibitor patients | 14% | (2/14) |
| FLT3-Unmutated (WT) | 10% | (4/39) |
| Patients | | |
| Venetoclax Naïve patients | 19% | (3/16) |
| Prior Venetoclax patients | 4% | (1/23) |

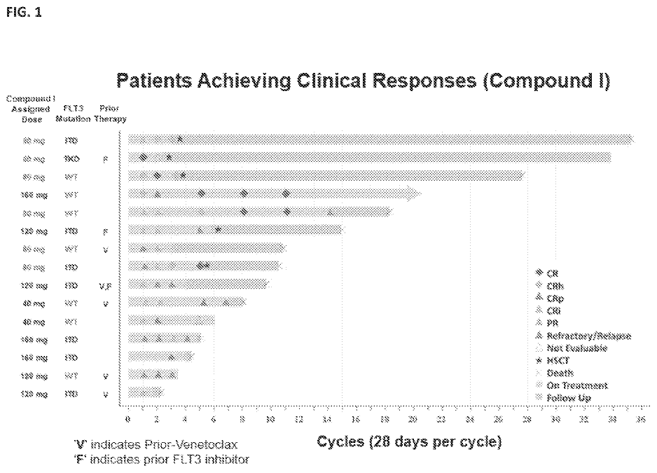

The data in Table 3 thus indicates that Compound I is particularly active in venetoclax naïve R/R AML patients than those with prior Venetoclax treatment. Table 3 also indicates that Compound I is active in FLT-3 WT and FLT-3 mutated AML patients. Compound I as a monotherapy has in fact been shown to achieve a clinical response to AML patients with FLT-3 WT and FLT-3 mutated patients, such as ITD or TKD FLT-3 mutations. FIG. 1, for example, shows a broad population of such patients that are responsive with doses as low as 80 mg once daily.

In addition to its activity, the administration of Compound I as a monotherapy is well tolerated. In fact, in the treated patients as indicated in Table 3 and FIG. 1, there were no treatment related QT_{c} prolongation, CPK elevations, differentiation syndrome, non-hematologic SAEs, or deaths of any patients in this study.

The study as described in Example 1 is also effective in the Compound I plus venetoclax combination treatment group. FIG. 2 shows a treatment plan with this combination. Specifically, Compound I/venetoclax combination is well tolerated and active in broad populations of relapsed or refractory (R/R) AML. Compound I/venetoclax also provides a unique opportunity due to its activity towards prior-venetoclax AML, including both FLT3^{MUT} and FLT3^{WT} AML in the R/R setting. Table 4 shows a broad population of such patients that are responsive when treated with the combination of Compound I and venetoclax.

TABLE 4
Compound I and Venetoclax Combination
Study Response Rates in R/R AML Patients
Compound I Composite Complete Remission (CRc)

| | |
|---|---|
| Subgroups | CRc Response Rate (n = 36) |

| Overall | 25% | (9/36)) |
|---|---|---|
| Venetoclax Naïve patients | 43% | (3/7) |
| Prior Venetoclax patients | 21% | (6/29) |
| FLT3-Mutated Patients | 36% | (4/11) |
| Venetoclax Naïve patients | 100% | (1/1) |
| Prior Venetoclax patients | 30% | (3/10) |
| Prior FLT3 inhibitor patients | 44% | (4/9) |
| FLT3-Unmutated (WT) | 20% | (5/25) |
| Patients | | |
| Venetoclax Naïve patients | 33% | (2/6) |
| Prior Venetoclax patients | 16% | (3/19) |

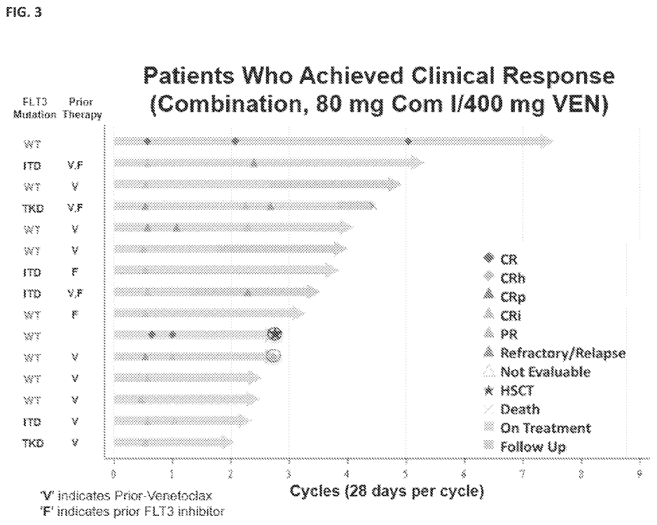

The data in Table 4 indicates that the combination of Compound I with Venetoclax is active towards broad populations of R/R AML, including activity towards FLT-3 WT patients and prior Venetoclax AML patients. FIG. 3 also shows a wide array of such patients that are responsive with doses as of 80 mg Compound I in combination with 400 mg Venetoclax. This includes FLT-3 WT and FLT-3 mutated patients, such as ITD or TKD FLT-3 mutations in FLT-3, and patients with prior-Venetoclax or prior FLT-3 inhibitor.

In addition to its activity, the administration of Compound I in combination with Ventoclax is well tolerated. In fact, in the treated patients as indicated in Table 4 and FIG. 3, the SAE events due to combination treatment was low at about 14%.

The combination of Compound I with Venetoclax administered to human patients thus indicates that the treatment is well tolerated and active in broad populations of R/R AML. Indeed, the combination shows activity towards patients that are FLT3^{WT} AML and FLT3^{MUT} AML with prior FLT3 inhibitor, and is active in both venetoclax naïve and prior venetoclax R/R AML, a known and very difficult patient population to treat.

1. A method of treating cancer in a subject in need thereof, comprising administering to the subject a therapeutically effective amount of compound of Formula (1), or a pharmaceutically acceptable salt thereof, solvate thereof, stereoisomer thereof, tautomer thereof, or combination thereof, wherein the subject has a mutant form of NPM1, DNMT3A, RAS, or a combination thereof: 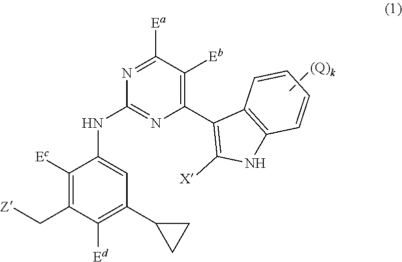
wherein in Formula (1):
Ea is hydrogen, hydroxy or C1-4 alkoxy;
Eb is hydrogen, halogen, C1-4 alkyl or C1-4 fluoroalkyl;
Ec and Ed are each independently hydrogen or hydroxy;
X′ is hydrogen or hydroxy;
k is an integer from 1 to 2;
each Q is independently hydroxy, halogen, C1-4 alkyl, hydroxy C1-4 alkyl or C1-4 alkoxy;
Z′ is a monovalent functional group represented by Formula (2); 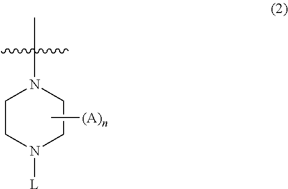
wherein:
each A is independently selected from the group consisting of hydroxy, C1-4 alkyl and hydroxy C1-4 alkyl, wherein at least one A is C1-4 alkyl;
n is an integer from 1 to 2; and
L is hydrogen, C1-4 alkyl, hydroxy or hydroxy C1-4 alkyl.

2. The method of claim 1, wherein the compound of Formula (1) is a compound of Formula (3), or a pharmaceutically acceptable salt thereof, solvate thereof, stereoisomer thereof, tautomer thereof, or combination thereof; 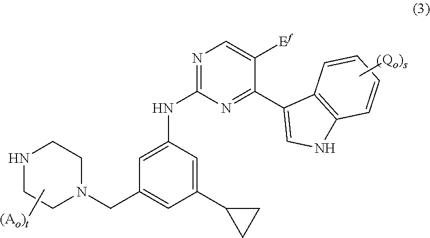
wherein in Formula 3:
Ef is fluorine, chlorine, bromine or iodine;
Qo is hydroxy, halogen, C1-4 alkyl, hydroxy C1-4 alkyl or C1-4 alkoxy;
s is an integer from 1 to 2;
Ao is selected from the group consisting of hydroxy, C1-4 alkyl and hydroxy C1-4 alkyl; and
t is an integer from 1 to 2.

3. The method of claim 1 or 2, wherein the compound of Formula (1) is Compound I 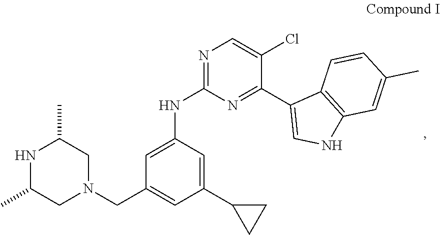
or a pharmaceutically acceptable salt thereof, solvate thereof, stereoisomer thereof, tautomer thereof, or combination thereof.

4. The method of any one of claims 1-3, wherein the subject additionally has a mutant form of FLT3.

5. The method of claim 4, wherein the subject has a mutant form of FLT3, a mutant form of NPM1, a mutant form of DNMT3A and a mutant form of RAS.

6. The method of any one of claims 1-5, wherein the subject has a mutant form of NRAS.

7. The method of any one of claims 1-5, wherein the subject has a mutant form of KRAS.

8. The method of claim 4, wherein the subject has a mutant form of FLT3 and a mutant form of NPM1.

9. The method of claim 4, wherein the subject has a mutant form of FLT3, a mutant form of NPM1 and a mutant form of DNMT3A.

10. The method of any one of claims 1-3, wherein the subject has a mutant form of RAS.

11. The method of claim 10, wherein the subject has a mutant form of NRAS.

12. The method of claim 10, wherein the subject has a mutant form of KRAS.

13. The method of any one of claims 10-12, wherein the subject further comprises a mutant form of FLT3.

14. The method of any one of claims 1-10 or 13, wherein the mutated FLT3 comprises an internal tandem duplication (ITD) mutation and/or at least one FLT3 point mutation.

15. The method of claim 14, wherein the at least one point mutation is on one or more residues selected from the group consisting of D835, F691, K663, Y842 and N841.

16. The method of any one of claims 1-10 or 13 wherein the mutated FLT3 comprises at least one mutation in the tyrosine kinase domain of FLT3.

17. The method of any one of claims 1-10 or 13, wherein the mutated FLT3 comprises an internal tandem duplication (ITD) mutation.

18. The method of any one of claims 1-10 or 13, wherein the mutated FLT3 comprises at least one point mutation is in the activation loop of FLT3.

19. The method of any one of claims 1-18, wherein the subject comprises one or mutations selected from the group consisting of NPM1-DNMT3A-FLT3-ITD, NRAS-ITD, KRAS-NPM1-DNMT3A-FLT3-ITD, NRAS-FLT3-ITD, NRAS-FLT3-WT, NPM1-FLT3-ITD,

20. The method of any one of claims 1-19, wherein the cancer is a hematological cancer.

21. The method of claim 20, wherein the hematological cancer is leukemia.

22. The method of claim 21, wherein the leukemia is acute myeloid leukemia (AML).

23. The method of claim 22, wherein the AML is relapsed or treatment-refractory (R/R) AML.

24. The method of any one of claims 1-23, wherein the subject has failed prior therapy with chemotherapy, hematopoietic stem cell transplantation (HSCT) therapy and/or therapy with other FLT3 inhibitors.

25. The method of any one of claims 1-24, wherein the compound of Formula (1) is formulated as a pharmaceutical formulation further comprising a pharmaceutically acceptable excipient.

26. The method of claim 3, wherein Compound I is administered in an amount of about 10 mg to about 300 mg.

27. The method of claim 26, wherein Compound I is administered in an amount of about 40 mg to about 160 mg.

28. The method of claim 26, wherein Compound I is administered in an amount of about 80 mg, about 120 mg, and/or about 160 mg.

29. The method of any one of claims 26-28, wherein the dosage amounts are administered once a day, twice a day, three times a day or four times a day.

30. The method of any one of claims 1-29, wherein the compound of Formula (1) is administered orally or parenterally.

31. A method of treating cancer in a subject in need thereof, comprising administering to the subject a therapeutically effective amount of Compound I, 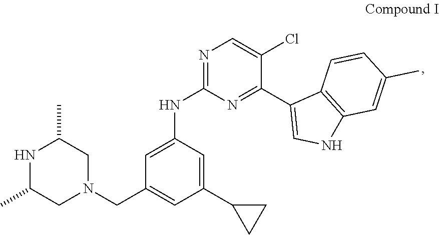
or a pharmaceutically acceptable salt thereof, solvate thereof, stereoisomer thereof, tautomer thereof, or combination thereof, wherein the cancer is relapsed or treatment-refractory (R/R) acute myeloid leukemia (AML).

32. The method of claim 31, wherein the subject has wildtype or a FLT3 mutation.

33. The method of claim 32, wherein the FLT3 mutation is a ITD or TKD mutation.

34. The method of claim 32, wherein the mutated FLT3 comprises an internal tandem duplication (ITD) mutation and/or at least one FLT3 point mutation.

35. The method of claim 32, wherein the at least one point mutation is on one or more residues selected from the group consisting of D835, F691, K663, Y842 and N841.

36. The method of any one of claims 32-35, wherein the mutated FLT3 comprises at least one mutation in the tyrosine kinase domain of FLT3.

37. The method of any one of claims 32-36, wherein the mutated FLT3 comprises an internal tandem duplication (ITD) mutation.

38. The method of any one of claims 32-37, wherein the mutated FLT3 comprises at least one point mutation is in the activation loop of FLT3.

39. The method of any one of claims 31 to 38, wherein the subject had prior BCL-2 inhibitor therapy or FLT3 inhibitor therapy.

40. The method of claim 39, wherein the BCL-2 inhibitor is venetoclax.

41. The method of claim 39, wherein the FLT3 inhibitor is lestaurtinib, sorafenib, midostaurin, quizartinib, crenolanib, and/or gilteritinib.

42. The method of any one of claims 31-41, wherein the dose of compound I is between about 20 mg to about 250 mg.

43. The method of claim 42, wherein the dose is administered orally to the subject once daily.

44. The method of claim 42 or 43, wherein the dose of Compound I or a pharmaceutically acceptable salt thereof, solvate thereof, stereoisomer thereof, tautomer thereof, or combination thereof, is about 40 mg, about 80 mg, about 120 mg, about 160 mg, or about 200 mg.

45. The method of any one of claims 31-44, wherein the dosing is administered once daily for 28 days.

46. The method of claim 45, wherein the 28 days of administration is a cycle that is repeated more than once, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times or more than 20 times to the subject.

47. The method of any one of claims 31-45, wherein Compound I, or a pharmaceutically acceptable salt thereof, solvate thereof, stereoisomer thereof, tautomer thereof, or combination thereof, is administered in combination with a BCL-2 inhibitor.

48. The method of claim 47, wherein the BCL-2 inhibitor is venetoclax.

49. The method of claim 47 or 48, wherein the combination treatment schedule is as described in FIG. 2.

50. The method of claim 47 or 48, wherein Compound I, or a pharmaceutically acceptable salt thereof, solvate thereof, stereoisomer thereof, tautomer thereof, or combination thereof, is administered once daily and the BCL-2 inhibitor or venetoclax is administered once daily.

51. The method of any one of claims 47-50, wherein Compound I or a pharmaceutically acceptable salt thereof, solvate thereof, stereoisomer thereof, tautomer thereof, or combination thereof, is administered at about 80 mg.

52. The method of any one of claims 48-51, wherein venetoclax is administered at a daily dose of about 100 mg to about 500 mg.

53. The method of any one of claims 48-51, wherein venetoclax is administered at a daily dose of about 400 mg.

54. The method of any one of claims 48-51, wherein venetoclax is administered at a lower daily dose at day 1 or day 2 of a treatment plan, with an increased daily dose at day 3, day 4, day 5, day 6, day 7, day 8, day 9 or day 10.

55. The method of any one of claims 1-54, wherein the subject is additionally administered a DNA hypomethylating agent (HMA).

56. The method of claim 55, wherein the HMA is selected from one or more of the group consisting of azacitidine, decitabine, and combinations thereof.
